(19) United States
(12) Patent Application Publication
(10) Pub. No.: US 2024/0368271 A1
(43) Pub. Date: Nov. 7, 2024

(54) Targeting GPR65 for the Treatment of Cancer in Overweight and Obese Individuals

(71) Applicant: The Board of Trustees of the Leland Stanford Junior University, Stanford, CA (US)

(72) Inventors: Edgar George Engleman, Atherton, CA (US); Sreya Bagchi, Redwood City, CA (US)

(21) Appl. No.: 18/683,174

(22) PCT Filed: Aug. 18, 2022
(86) PCT No.: PCT/US2022/075126

§ 371 Date: Feb. 12, 2024

Related U.S. Application Data
(60) Provisional application No. 63/234,524, filed on Aug. 18, 2021.

(51) Int. Cl. C07K 16/28; A61K 31/472; A61P 35/00; C12N 15/113

(52) U.S. Cl. CPC C07K 16/28; A61K 31/472; A61P 35/00; C12N 15/1138; C12N 2310/11; C12N 2310/14

(57) ABSTRACT

Currently, more than 60% of the U.S. population is overweight. and close to 40% is obese. Obesity is considered to be an epidemic. Obesity is not just characterized by an increase in body mass. but it also comes with a range of physiological changes. As a result. obese and ovenweight people are at a higher risk for developing life-threatening conditions such as cardiovascular diseases. diabetes and many types of cancers. Herein are methods. compositions and kits for enhancing immune cell mediated cancer treatment in overweight and obese individuals.

### CROSS-REFERENCE

This application claims the benefit of U.S. Provisional Patent Application No. 63/234,524filed Aug. 18, 2021, which application is incorporated herein by reference in its entirety.

### BACKGROUND

Currently, more than 60% of the U.S. population is overweight, and close to 40% is obese. Obesity is considered to be an epidemic. Obesity is not just characterized by an increase in body mass, but it also comes with a range of physiological changes. As a result, obese and overweight people are at a higher risk for developing life-threatening conditions such as cardiovascular diseases, diabetes and many types of cancers. For example, studies have shown that obese and overweight individuals face a higher chance of developing cancers of the endometrium, ovaries, kidney, gallbladder, breast, liver, pancreas, esophagus and colon. Not only does obesity increase the chance of developing a range of cancers, but it also leads to worse disease prognosis. In a large study, involving more than 900,000 adults in the US, it was discovered that obese men with cancers had a death rate that was 52% higher when compared to men of normal weight. Similarly, obese women with cancers had a death rate that was 62% higher when compared to normo-weight women. Given these correlations, it is important to investigate how deaths in cancer patients who are obese can be prevented.

It is now appreciated that immune cells can not only aggravate obesity-associated diseases, but also potentiate an inflammatory response in obese individuals, which is a major underlying cause of obesity-related co-morbidities. While the immune system consists of many different types of cells, the first responders that kickstart the process of inflammation are macrophages. In the context of tumors, macrophages and other immune cells often play an immunosuppressive tumor-promoting role.

In recent times, the importance of immune cells in cancer treatment has come into the spotlight as a result of the great success of immunotherapies, especially checkpoint blocking antibodies. These therapies do not target cancer cells directly but boost the body's immune cells that then kill the cancer cells. Most FDA approved cancer immunotherapies, including antibodies to checkpoints such as PD-1, PD-L1 and CTLA-4, target immune cells called T cells, which, when activated, exhibit potent anti-tumor properties. Unfortunately, a range of solid tumors lack T cells in the tumor microenvironment, which is believed to be one of the reasons why immunotherapies are not effective against a majority of solid tumors. Yet, even when T cells are absent from tumors, macrophages are present and can promote tumor growth and metastasis. In fact, they are one of the most abundant immune cells within solid tumor. Not surprisingly, macrophages have emerged as a promising, not yet proven, target for new immunotherapies. Approaches include both eliminating macrophages and reprogramming them to unleash their anti-tumor functions as opposed to their tumor promoting roles.

### SUMMARY

Compositions and methods are provided for the improved treatment of cancer in overweight and obese individuals. It is shown herein that tumor-associated macrophages in overweight and obese individuals overexpress GPR65, and that this overexpression reduces the efficacy of anti-tumor responses by these macrophages. Decreasing GPR65 activity increases the effectiveness of these macrophages. In obese and overweight persons, although macrophages tend to be inflammatory, surprisingly, macrophages in the tumors of obese subjects are less inflammatory and more immunosuppressive than are macrophages in the tumors of normal-weight subjects. These individuals can have a greater benefit from methods of enhancing anti-tumor responses by macrophages, relative to individuals of normal weight. In some embodiments, tumor associated macrophages in an overweight or obese individual have increased inflammatory activity after treatment with an inhibitor of GPR65, which may be manifest, for example and without limitation, by increased secretion of TNFa, increased interferon responses, increased expression of cytokines, decreased cancer cell growth, etc.

In some embodiments, methods are provided for the treatment of cancer in an obese individual, comprising administering an effective dose of an agent that blocks GPR65 activation. In some embodiments the agent is an antibody that selectively binds to GPR65 and prevents or inhibits signals through GPR65. In some embodiments the agent is a small molecule drug that selectively binds to GPR65 and inhibits GPR65 activation. In some embodiments administration is systemic. In some embodiments administration is localized to the tumor environment.

In some embodiments, methods are provided for the treatment of cancer in an obese individual, comprising administering an effective dose of an agent that decreases GPR65 expression in tumor-associated macrophages. In some embodiments the agent is an RNAi specific for GPR65. In some embodiments the agent is an anti-sense RNA specific for GPR65. In some embodiments administration is systemic. In some embodiments administration is localized to the tumor environment.

In some embodiments, methods are provided for the treatment of cancer in an obese individual, comprising administering an effective dose of macrophages deficient in GPR65 expression for delivery to tumors. For example, a macrophage-CARs cell, in which a tumor-tageting moiety is expressed on the either the patient's or another person's macrophages using gene delivery can be administered. In some embodiments the macrophage are engineered ex vivo to knock out or otherwise decrease GPR65 expression. In some embodiments the macrophages are autologous. In some embodiments the macrophages are allogeneic.

In some embodiments an anti-GPR65 agent for use in the methods disclosed herein is an antibody that specifically binds to an epitope in the first extracellular domain of human GPR65,e.g. an epitope within about residues 72-88 of the human protein, and blocks activation of GPR65.It is believed that acid-sensing capacity is mediated by proton transfer to histidines in the first extracellular loop of GPR65.

In some embodiments, methods are provided for the treatment of cancer in an individual selected for treatment agnostic of weight, i.e. including individuals having a BMI of <25 kg/m², the method comprising administering an effective dose of an antibody specific for the first extracellular loop of human GPR65, which antibody blocks GPR65 activation. In some embodiments administration is systemic. In some embodiments administration is localized to the tumor environment.

Various cancers can be treated with the methods disclosed herein. The cancer may be associated with obesity. The cancer may be a hematologic cancer, including without limitation multiple myeloma, lymphoma, and the like. The cancer may be a solid tumor, including without limitation carcinomas, meningiomas, melanomas, sarcomas, and the like. The cancer may be a breast carcinoma, hepatocellular carcinoma, pancreatic carcinoma, adenocarcinoma of the esophagus, uterine carcinoma, ovarian carcinoma, gall bladder carcinoma, carcinoma of the upper stomach, colorectal carcinoma, and the like. In some embodiments the cancer is a colorectal carcinoma.

In some embodiments, an individuals to be treated with the methods disclosed herein is overweight, having a body mass index (BMI) of ≥25 kg/m², BMI ≥26, BMI ≥27, BMI ≥28, BMI ≥29; or obese with a BMI ≥30 kg/m², BMI ≥31, BMI ≥32, BMI ≥33, BMI ≥34, BMI ≥35, or more. Tumor associated macrophages from a candidate patient may be evaluated prior to treatment to determine if there is an upregulation of GPR65 expression relative to a normal control or reference. Increased expression, e.g. greater than about 10%, 25%, 50%, 75% or more relative to a normal control can indicate a patient is suitable for treatment with the methods described herein.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. Gpr65 expression in mouse tissues under steady state. Cells from the blood, spleen and colon were isolated and stained with antibodies against myeloid cells, lymphocytes and CD45 non-immune cells (in the colon). Gpr65 expression on these cells was determined by green fluorescent protein signal (surrogate marker for Gpr65 expression), which was read by a flow cytometer. N=4

FIGS. 2A-2B. Expression of Gpr65 on myeloid cells in tumors of normal and obese mice. A) Myeloid cells were isolated from MC38 colon carcinoma tumors of normal (normal diet) and obese (high-fat diet) mice. Gpr65 expression on these cells was determined by green fluorescent protein signal (surrogate marker for Gpr65 expression), which was detected by a flow cytometer. B) Myeloid cells were isolated from the spleen of normal (normal diet) and obese (high-fat diet) mice. Gpr65 expression on these cells was determined by green fluorescent protein signal (surrogate marker for Gpr65 expression), which was read by a flow cytometer. N=3-5. A t-test was performed to detect statistically significant differences. ***p<0.005

FIGS. 3A-3B. Absence of Gpr65 reduces colon tumor growth in two different tumor models in obese mice. A) MC38 subcutaneous tumor volume was measured over a 2-week period in Gpr65 sufficient (Gpr65^{+/+}) and deficient (Gpr65^{-/-}) mice that were either normal (normal diet) or obese (high-fat diet). N=4-10. B) Tumor cells were injected into the colon of mice guided by a colonoscope. 3 weeks later, mice were euthanized, and tumors resected from the colon and weighed. N=2-10. A t-test was performed to detect statistically significant differences. *** p<0.005

FIG. 4. Tumor-associated macrophages in obese Gpr65 deficient mice secrete more TNF-α compared to obese Gpr65 sufficient mice. Subcutaneous MC38 colon tumors were resected from mice and the macrophages isolated and stained for production of TNF-α, a potent anti-tumor cytokine. Cytokine secretion was detected by flow cytometry. N=12-14. A t-test was performed to detect statistically significant differences. * p<0.05

FIGS. 5A-5C. Gpr65 expression in tumors is higher not only in obese patients with colon carcinoma, but also in obese patients with hepatocellular carcinoma and melanoma. GPR65 mRNA expression was determined by analyzing data available in the public GDC portal. (A) Colon carcinoma, (B) hepatocellular carcinoma and (C) melanoma RNA data was analyzed from both normal weight and obese individuals. A t-test was performed to detect statistically significant differences. * p<0.05, *** p<0.005.

FIG. 6: Ability of commercially available polyclonal antibody to inhibit GPR65 signaling. HEK293 cells or HEK293 cells transduced with human GPR65 (HEK293-hGPR65) were incubated in neutral pH (7.4) or acidic pH (6.8) without or with the polyclonal antibody at various concentrations for 30 minutes at 37° C. in a non-CO₂ incubator. Intracellular CAMP levels were subsequently determined using the Lance Ultra CAMP detection kit by PerkinElmer as per manufacturer's protocol.

FIG. 7: Ability of commercially available polyclonal antibody to inhibit GPR65 signaling. HEK293 cells or HEK293 cells transduced with human GPR65 (HEK293-hGPR65) were incubated in neutral pH (7.4) or acidic pH (6.8) without or with the polyclonal antibodies at various concentrations for 30 minutes at 37° C. in a non-CO₂ incubator. Intracellular CAMP levels were subsequently determined using the Lance Ultra CAMP detection kit by PerkinElmer as per manufacturer's protocol.

FIGS. 8A-8B: Ability of a small molecule to inhibit GPR65 signaling. A. HEK293 cells or HEK293 cells transduced with human GPR65 (HEK293-hGPR65) were incubated in neutral pH (7.4) or acidic pH (6.8) without or with the small molecule at various concentrations for 30 minutes at 37° C. in a non-CO₂ incubator. Intracellular cAMP levels were subsequently determined using the Lance Ultra CAMP detection kit by PerkinElmer as per manufacturer's protocol. B. Small molecule inhibitor structure.

FIG. 9: Ability of small molecule inhibitor to rescue TNF-α production from macrophages under acidic conditions. J774A.1 macrophage cells expressing human GPR65 (J774A.1-hGPR65) were exposed to either neutral pH or acidic pH overnight. The next day cells were treated with either vehicle control or the small molecule inhibitor shown in FIG. 8B, at a concentration of 10 μM in the presence of 100 ng/ml LPS for 2 hours. Cells were then fixed and permeabilized and stained for intracellular cytokine TNF-α.

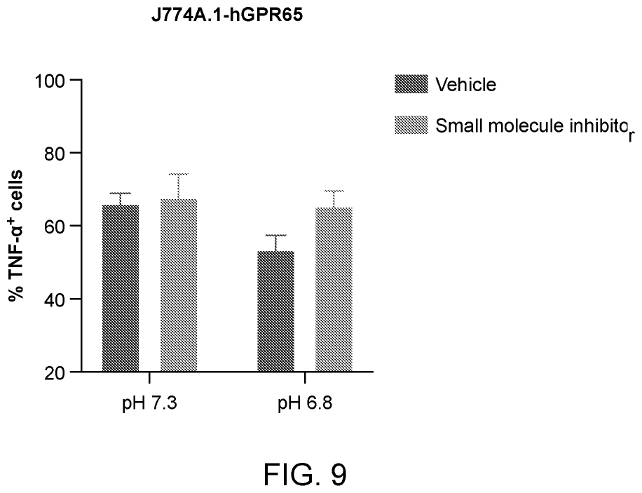

### DETAILED DESCRIPTION

Before the present methods and compositions are described, it is to be understood that this invention is not limited to the particular method or composition described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

GPR65. GPR65 is a G protein-coupled receptor (GPCR) protein that in humans is encoded by the GPR65 gene, also referred to as TDAG8. GPR65 (TDAG8) is primarily expressed in lymphoid tissues (spleen, lymph nodes, thymus, and leukocytes). As a GPCR, the protein is localized to the plasma membrane, with 7 transmembrane domains. The protein reference sequences for human GPR65 include Genbank NP_003599 and NP_032178.

It has been reported that 3- [(2,4-dichlorophenyl) methylsulfanyl]-1,6-dimethylpyridazino [4,5-e] [1,3,4] thiadiazin-5-one (referred to as BTB09089) is a partially specific agonist for GPR65. [(S)-phenyl (pyridin-4-yl) methyl] 4-methyl-2-pyrimidin-2-yl-1,3-thiazole-5-carboxylate (referred to as ZINC62678696) acts as a BTB09089 negative allosteric modulator. The small molecule PTT-3213 has been reported to be a selective inhibitor.

GPR65 senses extracellular pH by protonation of histidine residues on its extracellular domain, and when activated, GPR65 enables the downstream signaling through the Gq/11, Gs, and G12/13 pathways. Levels of cyclic adenosine monophosphate (cAMP) are found to be elevated in neutral to acidic extracellular pH (pH 7.0-6.5) in cells expressing GPR65, whereas in cells with mutated GPR65, this pH-sensing effect is reduced or eliminated. Overexpression of GPR65 can prevent tumor cell death in acidic conditions in vitro, and facilitates tumor growth in vivo. GPR65 reduces immune-mediated inflammation by regulating cytokine production of T cells (including IL-6, TNF-α and IL-1β) and macrophages.

Anti-GPR65 agent. As used herein, the term “anti-GPR65 agent” refers to any agent that blocks or inhibits GPR65 activity. Non-limiting examples of suitable anti-GPR65 reagents include GPR65 soluble polypeptides, anti-GPR65 antibodies or antibody fragments, anti-GPR65 small molecule drugs; and genetic agents that downregulate GPR65 expression, e.g. RNAi, anti-sense RNA, and may include genome reprogramming to reduce GPR65 expression. In some embodiments, a suitable anti-GPR65 agent specifically binds to and inhibits GPR65 without activating the receptor, e.g. by binding the extracellular portion of the GPR65 protein, including for example the amino terminal extracellular region of the protein, particularly including the first extracellular loop. A therapeutically effective dose is that dose that is sufficient to increase tumor associated macrophage inflammatory and anti-tumor activity, relative to untreated macrophages, e.g. a clinical indicia may be increased by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to the level in the absence of the agent.

The efficacy of a suitable anti-GPR65 agent can be assessed by assaying the agent. In an exemplary assay, target cells are incubated in the presence or absence of the candidate agent. An agent for use in the methods of the invention will block or inhibit GPR65 activity in the presence of an activating ligand such as protons, psychosine, etc., e.g. by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to the level in the absence of the agent.

In some embodiments, a therapeutically effective dose leads to sustained serum levels of anti-GPR65 agent (e.g., an anti-GPR65 antibody) of about 1 μg/ml or more (e.g, about 5 ug/ml or more, about 10 ug/ml or more, about 20 ug/ml or more, about 50 ug/ml or more, about 100 ug/ml or more). A therapeutically effective dose of an anti-GPR65 agent can depend on the specific agent used, but is usually about 0.1 mg/kg body weight, 0.5 mg/kg, 1 mg/kg, 2.5 mg/kg, 5 mg/kg, about 10 mg/kg or more, about 15 mg/kg or more, about 20 mg/kg or more, about 25 mg/kg or more, about 30 mg/kg or more, about 35 mg/kg or more, or about 40 mg/kg or more), or from about 10 mg/kg to about 40 mg/kg (e.g., from about 10 mg/kg to about 35 mg/kg, or from about 10 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

“Specific binding moiety”, as used herein, refers to an agent that interacts specifically to associate with a binding partner of interest, where the relative binding constant (Kd) is sufficiently strong to allow, for example, detection of binding to the partner of interest by a detection means; physiologically relevant association, etc. In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower KD.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

“Binding affinity” generally refers to the strength of the sum total of noncovalent interactions between a single binding site of a molecule (e.g., an antibody or other binding molecule) and its binding partner (e.g., an antigen or receptor). The affinity of a molecule X for its partner Y can generally be represented by the dissociation constant (Kd). Affinity can be measured by common methods known in the art, including those described herein. For example, low-affinity antibodies bind antigen (or receptor) weakly and tend to dissociate readily, whereas high-affinity antibodies bind antigen (or receptor) more tightly and remain bound longer.

Anti-GPR65 antibodies. In some embodiments, a subject anti-GPR65 agent is an antibody that specifically binds GPR65 and inhibits its activity. In some embodiments, a suitable anti-GPR65 antibody does not activate GPR65 upon binding. “Antibody” shall include, by way of example, both naturally occurring and non-naturally occurring antibodies. Specifically, this term includes polyclonal and monoclonal antibodies, and fragments thereof. Furthermore, this term includes chimeric antibodies and wholly synthetic antibodies, and fragments thereof, including multi-specific antibodies, such as bispecific antibodies. For example, in order to selectively deliver an anti-GPR65 antibody or a small molecule GPR65 inhibitor to the tumor, a bispecific antibody may be selected that targets both GPR65 and a tumor marker such as Her-2neu for breast cancer, CEA for colorectal cancer, and even PD-L1, which is expressed on both tumor cells and some TAMs. The latter would not only provide selective delivery of the GPR65 inhibitor, but also achieve blockade of PD-L1.

Suitable anti-GPR65 antibodies include fully human, humanized or chimeric versions of such antibodies. Humanized antibodies may be useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-GPR65 antibodies are known in the art and commercially available, e.g. human TDAG8/GPR65 Antibody, MAB10077; Anti-GPR65 monoclonal antibody (DCABH-15581); TRANS GENIC INC. Anti Human GPR65 Monoclonal Antibody (Clone No. 23D5); Anti Human GPR65 Monoclonal Antibody (Clone No. 11F1); and the like. Polyclonal antibodies include, for example, (Invitrogen) PA5-111835.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics. In certain embodiments an epitope of GPR65 is an extracellular region of the protein, including, for example and without limitation, the first extracellular loop.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light-and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and-binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113,Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, IgA₂. The heavy-chain constant domains that correspond to the different classes of immunoglobulins are called α, δ, γ, ε, and μ, respectively. The subunit structures and three-dimensional configurations of different classes of immunoglobulins are well known.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the lgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

Monoclonal antibodies include hybrid and recombinant antibodies produced by splicing a variable (including hypervariable) domain of an anti-CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Isolated” antibodies are polyclonal antibodies or monoclonal antibodies that have been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibodies will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibodies will be prepared by at least one purification step.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

Anti-GPR65 genetic agents. In some embodiments, an anti-GPR65 agent is an agent that that specifically downregulates GPR65 expression in a target call, e.g. RNAi or anti-sense RNA. An antisense reagent may be antisense oligonucleotides (ODN), particularly synthetic ODN having chemical modifications from native nucleic acids, or nucleic acid constructs that express such antisense molecules as RNA. The antisense sequence is complementary to GPR65, and inhibits its expression. One or a combination of antisense molecules may be administered, where a combination may comprise multiple different sequences.

Antisense molecules may be produced by expression of all or a part of the target GPR65 sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 25,usually not more than about 23-22 nucleotides in length, where the length is governed by efficiency of inhibition, specificity, including absence of cross-reactivity, and the like.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The.alpha.-anomer of deoxyribose may be used, where the base is inverted with respect to the natural.beta.-anomer. The 2′-OH of the ribose sugar may be altered to form 2′-O-methyl, 2′-Fluoro, or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively. Anti-sense molecules of interest include antagomir RNAs, e.g. as described by Krutzfeldt et al., herein specifically incorporated by reference. Antagomirs are cholesterol-conjugated single-stranded RNA analogs. Antagomir RNAs may be synthesized using standard solid phase oligonucleotide synthesis protocols. The RNAs are conjugated to cholesterol, and may further have a phosphorothioate backbone at one or more positions.

Also of interest in certain embodiments are RNAi agents. By RNAi agent is meant an agent that modulates expression of GPR65 mRNA by a RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. Where the RNA agent is a duplex structure of two distinct ribonucleic acids hybridized to each other, e.g., an sRNA, the length of the duplex structure typically ranges from about 15 to 30 bp, usually from about 15 to 29 bp, where lengths between about 20 and 29 bps, e.g., 21 bp, 22 bp, are of particular interest in certain embodiments. Where the RNA agent is a duplex structure of a single ribonucleic acid that is present in a hairpin formation, i.e., a shRNA, the length of the hybridized portion of the hairpin is typically the same as that provided above for the sRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

dsRNAi agents can be prepared according to any of a number of methods that are known in the art, including in vitro and in vivo methods, as well as by synthetic chemistry approaches. Examples of such methods include, but are not limited to, the methods described by Sadher et al. (Biochem. Int. 14:1015, 1987); by Bhattacharyya (Nature 343:484, 1990); and by Livache, et al. (U.S. Pat. No. 5,795,715), each of which is incorporated herein by reference in its entirety. Single-stranded RNA can also be produced using a combination of enzymatic and organic synthesis or by total organic synthesis. The use of synthetic chemical methods enable one to introduce desired modified nucleotides or nucleotide analogs into the dsRNA. dsRNA can also be prepared in vivo according to a number of established methods (see, e.g., Sambrook, et al. (1989) Molecular Cloning: A Laboratory Manual, 2nd ed.; Transcription and Translation (B. D. Hames, and S. J. Higgins, Eds., 1984); DNA Cloning, volumes I and II (D. N. Glover, Ed., 1985); and Oligonucleotide Synthesis (M. J. Gait, Ed., 1984, each of which is incorporated herein by reference in its entirety).

Examples of suitable RNAi targets include, for example:

TABLE 1

| | | Starting |
|---|---|---|
| | | position in |
| | RNAi targets in human GPR65 | NM_003608 |
| SEQ ID NO: 1 | TGAAGAACAGCATGACCTGGATCAC | 565 |
| SEQ ID NO: 2 | ACAGCATGACCTGGATCACTATTTG | 571 |
| SEQ ID NO: 3 | ATCTTTGTGATTATAGTCAGCATTC | 611 |
| SEQ ID NO: 4 | CAATATTGGATCTCTGTGTGTGTCT | 640 |
| SEQ ID NO: 5 | CTTCAGTTTGTCACTATCAGATTTA | 706 |
| SEQ ID NO: 6 | GTTGATCGGTATTTGGCTGTTGTCT | 875 |
| SEQ ID NO: 7 | CGATGCCGAAAAGTCTAATTTTACT | 1027 |
| SEQ ID NO: 8 | GAAGAGAATCATAAAACTACTTGTC | 1210 |
| SEQ ID NO: 9 | CACAGTTACTTTTGTCTTATGCTTT | 1240 |
| SEQ ID NO: 10 | TTGTCTTATGCTTTACTCCCTTTCA | 1251 |
| SEQ ID NO: 11 | GAGCATGCTGTGAACTTCGAAGACC | 1304 |
| SEQ ID NO: 12 | GCAATTCTGGGAAGCGAACTTACAC | 1332 |
| SEQ ID NO: 13 | CTGGGAAGCGAACTTACACAATGTA | 1338 |
| SEQ ID NO: 14 | GCATTAACAAGTTTAAATTGTGTTG | 1376 |
| SEQ ID NO: 15 | ATATGATATGTGGAATATATTAAAA | 1411 |
| SEQ ID NO: 16 | GTCACCGAGCTTGAAAGATTTCTAT | 2559 |

In certain embodiments, instead of the RNAi agent being an interfering ribonucleic acid, e.g., an sRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

Macrophages may be genetically altered in order to down-regulate GPR65, e.g. by introduction of an anti-sense or RNAi sequence specific for GPR65. Cells may be genetically altering by transfection or transduction with a suitable vector, homologous recombination, or other appropriate technique, so that they express a sequence of interest. Alternatively programmable gene editing tools such as CRISPR/cas9 and the like can be used to edit genomes to reduce GPR65 expression, e.g. by knocking out the sequence, or by introducing regulated expression of the protein or of an anti-sense or RNAi.

In some cases, a cell is modified by a class 2 CRISPR/Cas effector protein (or a nucleic encoding the protein), e.g., as an endonuclease. In class 2 CRISPR systems, the functions of the effector complex (e.g., the cleavage of target DNA) are carried out by a single protein (which can be referred to as a CRISPR/Cas effector protein)—where the natural protein is an endonuclease (e.g., see Zetsche et al, Cell. 2015 Oct. 22;163 (3): 759-71; Makarova et al, Nat Rev Microbiol. 2015 November;13 (11): 722-36; Shmakov et al., Mol Cell. 2015 Nov. 5;60 (3): 385-97; and Shmakov et al., Nat Rev Microbiol. 2017 March;15 (3): 169-182: “Diversity and evolution of class 2 CRISPR-Cas systems”). As such, the term “class 2 CRISPR/Cas protein” or “CRISPR/Cas effector protein” is used herein to encompass the effector protein from class 2 CRISPR systems—for example, type Il CRISPR/Cas proteins (e.g., Cas9), type V CRISPR/Cas proteins (e.g., Cpf1/Cas12a, C2c1/Cas12b, C2C3/Cas12c), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

In some cases, an RNA-guided endonuclease is a fusion protein that is fused to a heterologous polypeptide (also referred to as a “fusion partner”). In some cases, an RNA-guided endonuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.). In some embodiments, an RNA-guided endonuclease is fused to an amino acid sequence (a fusion partner) that provides a tag (i.e., the fusion partner is a detectable label) for ease of tracking and/or purification (e.g., a fluorescent protein, e.g., green fluorescent protein (GFP), YFP, RFP, CFP, mCherry, tdTomato, and the like; a histidine tag, e.g., a 6XHis tag; a hemagglutinin (HA) tag; a FLAG tag; a Myc tag; and the like). In some cases, the fusion partner can provide for increased or decreased stability (i.e., the fusion partner can be a stability control peptide, e.g., a degron, which in some cases is controllable (e.g., a temperature sensitive or drug controllable degron sequence).

An RNA-guided endonuclease (e.g., a Cas9 protein) can have multiple (1 or more, 2 or more, 3 or more, etc.) fusion partners in any combination of the above. As an illustrative example, an RNA-guided endonuclease (e.g., a Cas9 protein) can have a fusion partner that provides for tagging (e.g., GFP), and can also have a subcellular localization sequence (e.g., one or more NLSs). In some cases, such a fusion protein might also have a tag for ease of tracking and/or purification (e.g., a histidine tag, e.g., a 6XHis tag; a hemagglutinin (HA) tag; a FLAG tag; a Myc tag; and the like). As another illustrative example, an RNA-guided endonuclease (e.g., a Cas9protein) can have one or more NLSs (e.g., two or more, three or more, four or more, five or more, 1, 2, 3, 4, or 5 NLSs). In some cases a fusion partner (or multiple fusion partners, e.g., 1, 2, 3, 4,or 5 NLSs) (e.g., an NLS, a tag, a fusion partner providing an activity, etc.) is located at or near the C-terminus of the RNA-guided endonuclease (e.g., Cas9 protein). In some cases a fusion partner (or multiple fusion partners, e.g., 1, 2, 3, 4, or 5 NLSs) (e.g., an NLS, a tag, a fusion partner providing an activity, etc.) is located at the N-terminus of the RNA-guided endonuclease (e.g., Cas9 protein). In some cases the genome editing nuclease (e.g., Cas9 protein) has a fusion partner (or multiple fusion partners, e.g., 1, 2, 3, 4, or 5 NLSs) (e.g., an NLS, a tag, a fusion partner providing an activity, etc.) at both the N-terminus and C-terminus.

Vectors useful for transferring exogenous sequences, e.g. anti-sense or RNAi sequence specific for GPR65, into target mammalian cells are also available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc.

Nucleic acids are “operably linked” when placed into a functional relationship with another nucleic acid sequence. Expression vectors will contain a promoter that is recognized by the host organism and is operably linked to the anti-sense or RNAi sequence specific for GPR65.Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In other embodiments, macrophages are modified by introduction of an anti-sense or RNAi sequence specific for GPR65, where GPR65 is then expressed at decreased levels relative to non-modified macrophages, e.g. expressing at a level 5-fold less than unmodified cells, 10-fold less, 50-fold less, 100-fold less, or more. In some embodiments the RNA is modified RNA, e.g. comprising modified nucleosides, e.g. one or more of thiouridine, 5-methylcytidine (m5C), N1-methyl-pseudouridine (1mΨ), pseudouridine (Ψ), etc., particularly m5C and Ψ. For in vivo delivery, the modified RNA may be packaged in lipid nanoparticles, e.g. comprising ionizable lipids, phospholipids, and cholesterol, where the outside of the particle is coated in pegylated lipids.

### Small Molecule Inhibitors

Candidate agents of interest as anti-GPR65 agents are biologically active agents that encompass numerous chemical classes, primarily organic molecules, which may include organometallic molecules, inorganic molecules, genetic sequences, etc. Candidate drugs, select therapeutic antibodies and protein-based therapeutics, can be evaluated to determine binding and inhibition of GPR65. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and lons; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Test compounds can for screening purposes, e.g. to determine binding and inhibition of GPR65, include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 .mu.l to 1ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of cells, proteins, etc., usually in conjunction with wells lacking the agent. The change in parameter readout in response to the agent is measured, desirably normalized, and the resulting data may then be evaluated by comparison to reference data. Agents of interest for analysis include any biologically active molecule with the capability of binding and inhibition of GPR65.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Initial screening assays for binding and inhibition may be followed up by evaluation in cell systems.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

Treatment of colorectal carcinoma is of interest. Colorectal cancer accounts for an estimated 140,250 cases and 50,630 deaths in the US annually. Incidence rises sharply around age 40 to 50. Overall, more than half of the cases occur in the rectum and sigmoid, and 95% are adenocarcinomas. Colorectal cancer is slightly more common among men than women. Synchronous cancers (more than one) occur in 5% of patients. Colorectal cancer (CRC) most often occurs as transformation within adenomatous polyps. About 80% of cases are sporadic, and 20% have an inheritable component. Predisposing factors include chronic ulcerative colitis and Crohn colitis; the risk of cancer increases with the duration of these disorders. CRC spreads by direct extension through the bowel wall, hematogenous metastasis, regional lymph node metastasis, and perineural spread.

Newer drugs used singly or in drug combinations include capecitabine (a 5-fluorouracil precursor), irinotecan, and oxaliplatin. Monoclonal antibodies such as bevacizumab, cetuximab, and panitumumab are also being used with some effectiveness. No regimen is clearly more effective for prolonging life in patients with metastatic colorectal cancer, although some have been shown to delay disease progression.

Obesity. The term “obesity” means the condition of excess body fat (adipose tissue), including by way of example in accordance with the National Institutes of Health Federal Obesity Clinical Guidelines for adults, whereby body mass index (“BMI”) calculated by dividing body mass in kilograms by height in meters squared is equal to or greater than twenty-five (25). In general terms obesity denotes excess body fat; the consequences of which depend not only on the absolute amount but also on the distribution of the fat. Complications include cardiovascular disorders, diabetes mellitus, many cancers, cholelithiasis, fatty liver and cirrhosis, osteoarthritis, reproductive disorders in men and women, psychologic disorders, and premature death. Diagnosis is based on body mass index calculated from height and weight, and waist circumference. BP, fasting plasma glucose, and lipid levels should be measured. Treatment includes physical activity, dietary and behavioral modification, and sometimes drugs or surgery.

With aging, body fat increases and is redistributed to the abdomen, and muscle mass is lost, largely because of physical inactivity, but decreased androgens and growth hormone (which are anabolic) and inflammatory cytokines produced in obesity may also play a role.

It will be understood that there are medically accepted definitions of obesity and overweight. A patient may be identified by, for example, measuring body mass index (BMI), which is calculated by dividing weight in kilograms by height in meters squared, and comparing the result with the definitions. The recommended classifications for BMI in humans, adopted by the Expert Panel on the Identification, Evaluation and Treatment of Overweight and Obesity in Adults, and endorsed by leading organizations of health professionals, are as follows: underweight <18.5 kg/m², normal weight 18.5-24.9 kg/m², overweight 25-29.9 kg/m², obesity (class 1) 30-34.9 kg/m², obesity (class 2) 35-39.9 kg/m², extreme obesity (class 3) >40 kg/m², (Practical Guide to the Identification, Evaluation, and Treatment of Overweight and Obesity in Adults, The North American Association for the Study of Obesity (NAASO) and the National Heart, Lung and Blood Institute (NHLBI) 2000). Modifications of this classification may be used for specific ethnic groups.

Another alternative for assessing overweight and obesity is by measuring waist circumference. There are several proposed classifications and differences in the cutoffs based on ethnic group. For instance, according to the classification from the International Diabetes Federation, men having waist circumferences above 94 cm and women having waist circumferences above 80 cm are at higher risk of diabetes, dyslipidemia, hypertension and cardiovascular diseases because of excess abdominal fat. Another classification is based on the recommendation from the Adult Treatment Panel III where the recommended cut-offs are 102cm for men and 88 cm for women.

The terms “subject,” “individual,” and “patient” are used interchangeably herein to refer to a mammal being assessed for treatment and/or being treated. In some embodiments, the mammal is a human, e.g. an overweight or obese subject. The terms “subject,” “individual,” and “patient” encompass, without limitation, individuals having a disease, e.g. cancer. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent, e.g. an infusion of engineered cells, etc., sufficient to treat or manage a disease or disorder. A therapeutically effective amount may refer to the amount of therapeutic agent sufficient to delay or minimize the onset of disease, e.g., to delay or minimize the growth and wspread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4weeks, 5 weeks, 6 weeks, 8 weeks, or 12 weeks after) the administration of a second prophylactic or therapeutic agent to a subject with a disorder.

The GPR65 agent may be used alone or in combination with other therapeutic intervention such as radiotherapy, chemotherapy, immunosuppressant and immunomodulatory therapies.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinoIAQ (Cisplatin), PlatinoIAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### Cell Compositions

In some embodiments a macrophage cell composition is provided that has been genetically altered to reduce GPR65 expression, or is provided in combination with a GPR65 inhibitor agent. The cell can be provided in a unit dose for therapy, and can be allogeneic, autologous, etc. with respect to an intended recipient. Methods may include a step of obtaining desired cells, e.g., myeloid progenitor cells, hematopoietic stem cells, etc., which may be isolated from a biological sample, or may be derived in vitro from a source of progenitor cells. The cells can be modified to knock out or decrease GPR65 expression. For example, cells may be collected from a patient, modified ex vivo, and reintroduced into the subject. The cells collected from the subject may be collected from any convenient and appropriate source, including e.g., peripheral blood (e.g., the subject's peripheral blood), a biopsy (e.g., a biopsy from the subject), and the like.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The cells can be administered by any suitable means, usually parenteral. Parenteral infusions include intramuscular, intravenous (bolus or slow drip), intraarterial, intraperitoneal, intrathecal or subcutaneous administration.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLE 1

Targeting GPR65 for the treatment of cancer in overweight and obese individuals.

To identify the mechanism responsible for accelerated tumor growth and abnormal macrophage functioning under conditions of obesity, we started out by characterizing tumors and the macrophages in tumors of obese mice. Obesity in mice was modeled by feeding them a high-fat diet (60% kcal fat, 20% kcal protein, 20% kcal carbohydrate), while control mice were fed a normal chow diet consisting of 13% kcal fat, 34% kcal protein and 53% kcal carbohydrate. We chose to use the high-fat diet model of obesity because it is known that in humans, a combination of excessive caloric intake and the availability of energy dense meals is one of the major contributors to the development of obesity. In our initial studies, we confirmed that colorectal tumors grow significantly faster and larger in obese vs. normal mice. Thereafter, we investigated the tumor-associated macrophages (TAMs) in these mice and discovered that they are more abundant under conditions of obesity. Not only did obesity affect TAM numbers, but it also caused the them to display pro-tumorigenic activity in vivo, leading to faster tumor growth when compared to non-obese mice.

To understand why TAMs were more tumorigenic under conditions of obesity, we performed gene expression profiling on the TAMs. The results showed that these cells expressed higher levels of the cell surface protein GPR65. The upregulation of this receptor can explain why TAMs in obese mice are more pro-tumorigenic, since signals through this receptor blunt anti-tumor immune responses in macrophages. These results led us to test the utility of targeting Gpr65 for the treatment of cancer under conditions of obesity.

GPR65 was first described as a molecule that is upregulated during activation-induced apoptosis in T cells in the thymus. This led to the receptor being named T cell death associated gene 8 (TDAG8). It is a G protein coupled receptor (GPCR) expressed on cell surfaces and it senses extracellular acid. When it is bound by its ligands, intracellular signals are propagated through increased cyclic adenosine monophosphate (cAMP) and protein kinase A (PKA) activity, ultimately resulting in activation of the transcription factor cAMP responsive element modulator (CREM), which controls the transcription of many downstream genes. It plays an important role in inflammatory conditions involving the presence of extracellular acid. Signals through GPR65 keep autoimmune and allergic inflammation at bay (EAE, arthritis, colitis and delayed-type hypersensitivity response). These responses are mediated by GPR65 on macrophages, neutrophils, T cells and B cells (in the absence of GPR65, these diseases are exacerbated). GPR65 signaling is also known to blunt inflammatory responses in macrophages.

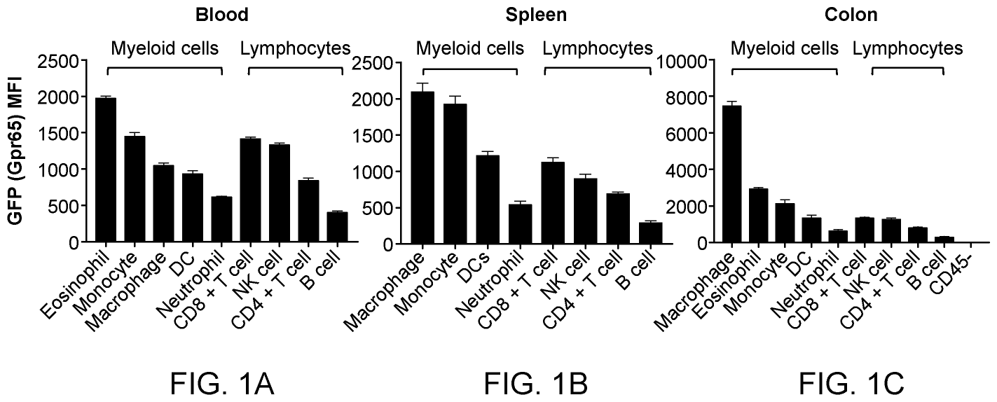

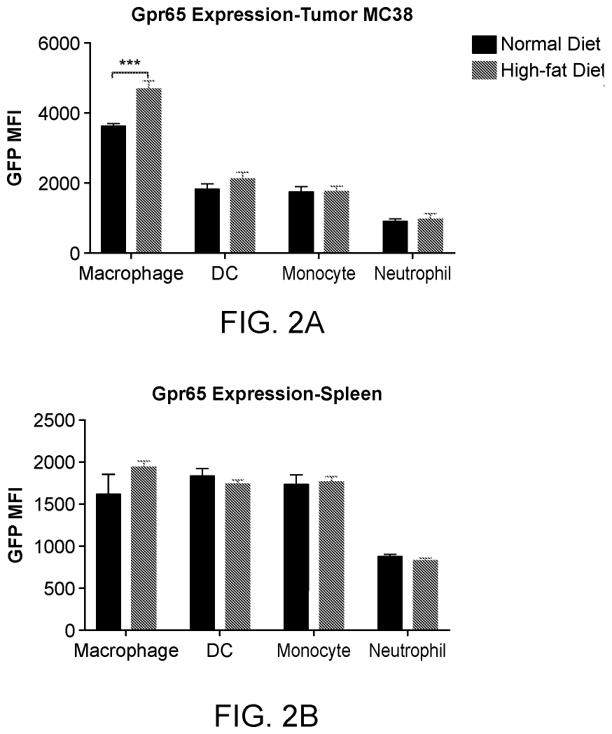

Targeting Gpr65 for the treatment of cancer under conditions of obesity. In mice, under steady state, Gpr65 is highly expressed by myeloid cells (macrophages, monocytes and eosinophils), T cells and NK cells in the blood, spleen and colon (FIG. 1). Gpr65 expression on TAMs is higher under conditions of obesity in mice. This increase is limited to TAMs and is not observed in other myeloid cells (FIG. 2a). The increase is also limited to the tumor, i.e. there is no difference in Gpr65 expression on macrophages and other myeloid cells in the spleen between normal and obese mice (FIG. 2b). Gpr65 expression is generally much higher in the tumor compared to the spleen (FIGS. 2a and b).

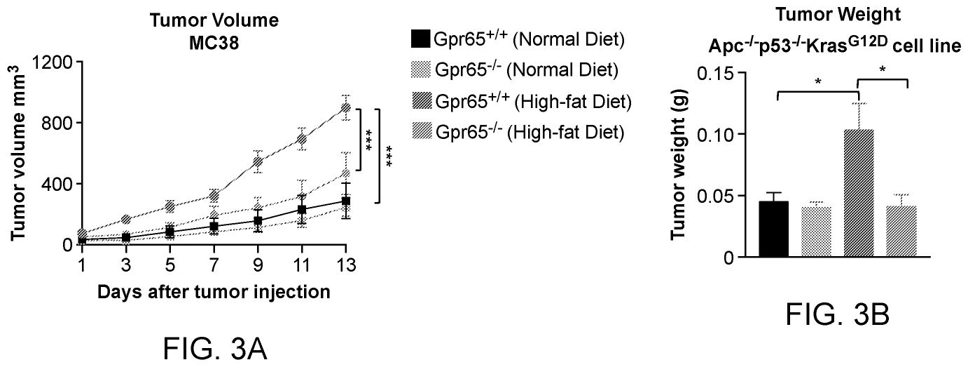

Selective absence of Gpr65 reduces colorectal cancer (CRC) burden in two different CRC models in obese mice (absence of Gpr65 is denoted by Gpr65 ^{-/-} and presence is denoted by Gpr65^{+/+}). The MC38 subcutaneous CRC cell line model is shown in FIG. 3a. The Apc^{-/-}p53^{-/-} Kras^{G12D} CRC cell line orthotopic model is shown in FIG. 3b.

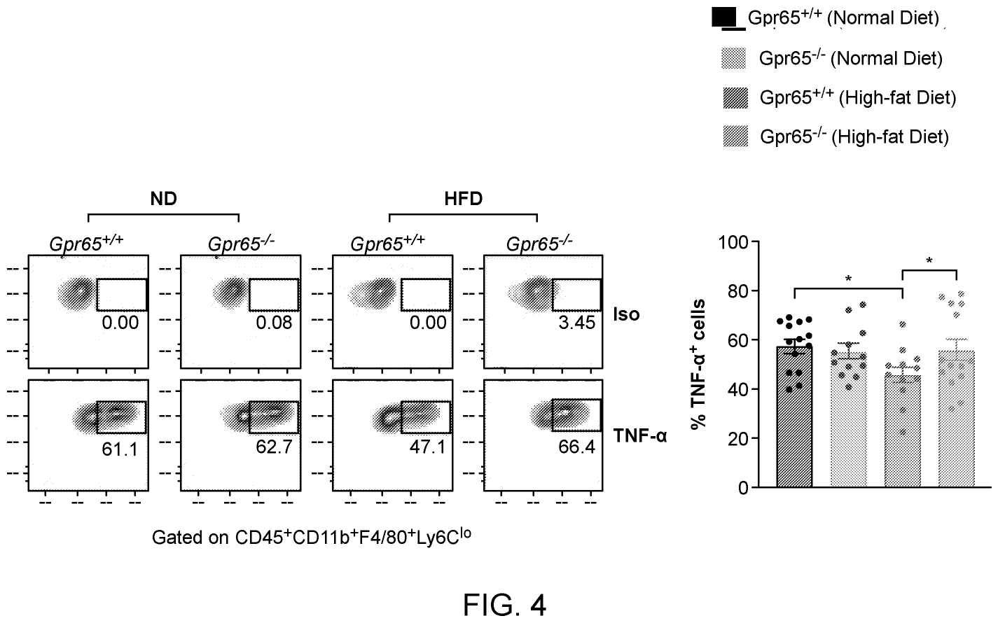

In mice lacking Gpr65, TAMs in MC38 tumors of obese mice secrete more TNF-α, an anti-tumor mediator (FIG. 4).

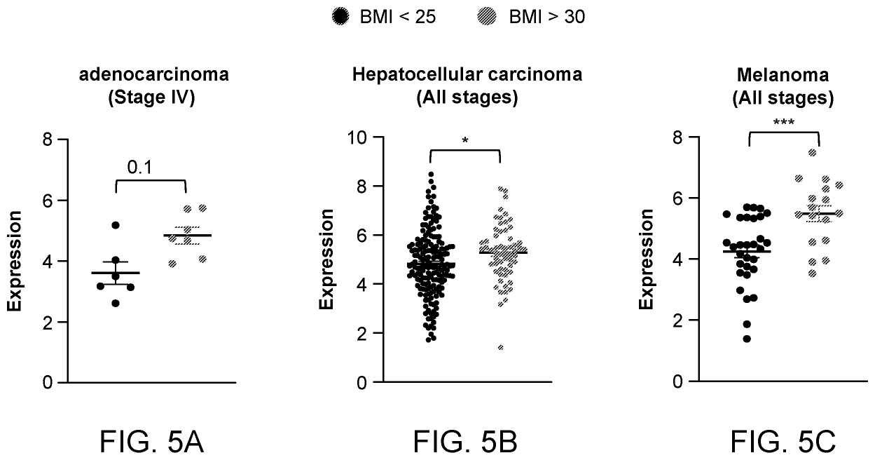

GPR65 expression is higher in human colon carcinomas, hepatocellular carcinomas and melanomas in obese individuals compared to normal weight individuals (FIG. 5). Targeting Gpr65 for cancer treatment has applicability in a number of cancers; including without limitation hepatocellular carcinoma and melanoma.

### EXAMPLE 2

Antibody Inhibition

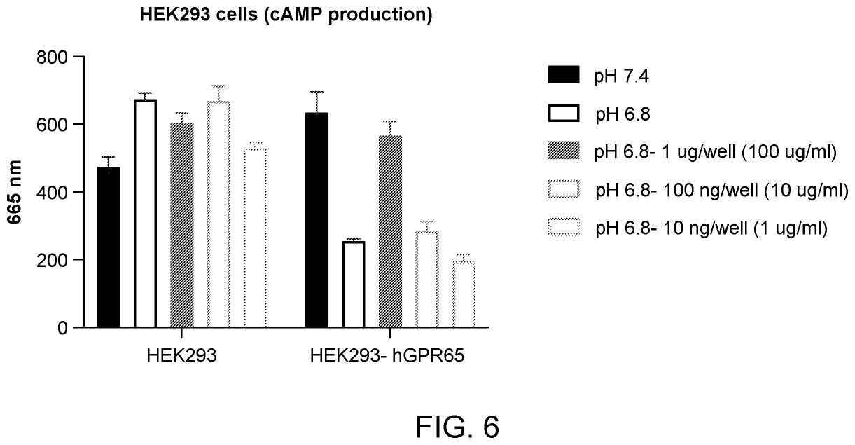

We tested the ability of a commercially available antibody to inhibit human GPR65signaling in the presence of its natural ligand, protons. To this end, we transduced HEK293 cells to express the receptor (HEK293-hGPR65). Both parental HEK293 cells and the transduced HEK293-hGPR65 cells were exposed to either neutral pH or acidic pH and intracellular CAMP levels quantified. A kit from PerkinElmer was used to quantify cAMP levels, where low readings at 665 nm suggest high CAMP levels and vice versa. As can be seen in FIG. 6, while CAMP production did not increase in the parental HEK293 cells when exposed to acidic medium, HEK293-hGPR65 cells exhibited increased CAMP production (as denoted by the lower OD reading) upon exposure to acidic medium. Further, this increased CAMP production was abrogated when HEK293-hGPR65 cells were exposed to both acidic medium and the highest concentration of the antibody (ThermoFisher (Invitrogen) PA5-111835). Upon lowering the antibody concentration, cAMP production increased in a dose dependent manner. These antibodies were specific for the 1st extracellular loop of the human GPR65 (amino acid residues 72-84.

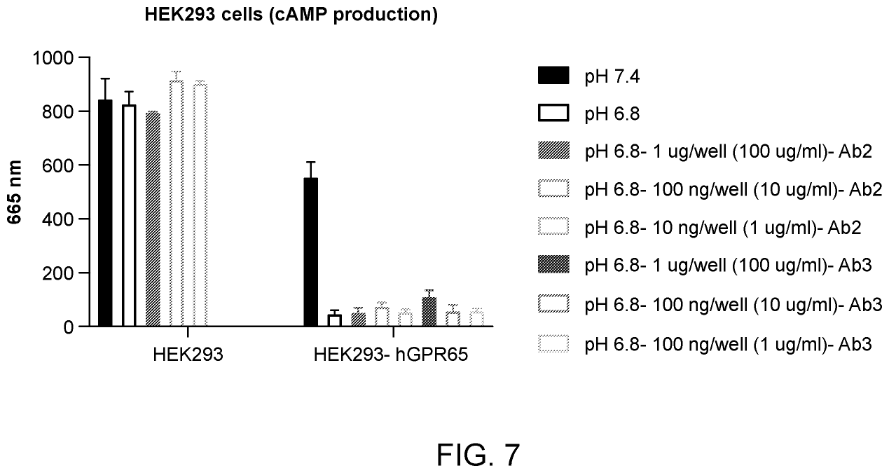

We also tested two other commercially available antibody clones. These did not show any inhibitory activity against GPR65 activity (FIG. 7). Antibodies were from Abnova (PAB26550), which is specific for an intracellular (cytoplasmc) domain and ThermoFisher (PA5-33753), specific for the 3rd extracellular domain.

### EXAMPLE 3

### Small Molecule Inhibition

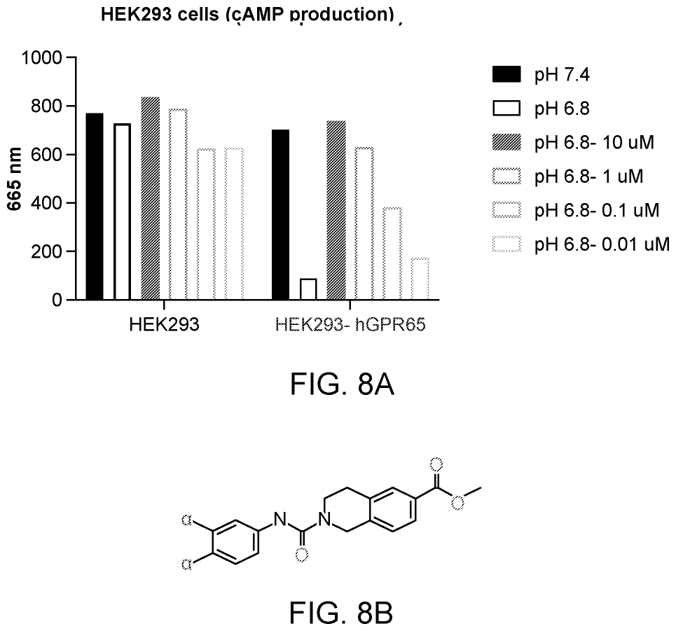

We tested the ability of a small molecule to inhibit human GPR65 signaling in the presence of its natural ligand, protons. To this end, we transduced HEK293 cells to express the receptor (HEK293-hGPR65). Both parental HEK293 cells and the transduced HEK293-hGPR65 cells were exposed to either neutral pH or acidic pH and intracellular cAMP levels quantified. A kit from PerkinElmer was used to quantify cAMP levels where low readings at 665 nm suggest high CAMP levels and vice versa. As can be seen in FIG. 8A, while cAMP production did not increase in the parental HEK293 cells when exposed to acidic medium, HEK293-hGPR65 cells exhibited increased CAMP production (as denoted by the lower OD reading) upon exposure to acidic medium. Further, this increased cAMP production was abrogated when HEK293-hGPR65 cells were exposed to both acidic medium and the highest concentration of the small molecule (structure shown in FIG. 8B). Upon lowering the small molecule concentration, cAMP production increased in a dose dependent manner.

To determine if the same small molecule inhibitor could affect cytokine production from macrophages in vitro, we transduced a macrophage cell line, J774A.1 to express human GPR65(J774A.1-hGPR65) and exposed the cells to both neutral and acidic conditions in the presence of vehicle control or the small molecule inhibitor. Since it is known that signaling through GPR65dampens production of TNF-α, we examined the ability of J774A.1-hGPR65 to produce the cytokine. As expected, TNF-α production was lower in vehicle treated cells under acidic conditions. However, blunted production of the cytokine was rescued when the cells were treated with the small molecule inhibitor. Lastly, there was no difference in TNF-α production by the cells in neutral pH treated with vehicle or the small molecule inhibitor.

### References

1. Blüher, M. Obesity: global epidemiology and pathogenesis. Nat Rev Endocrinol 15, 288-298(2019).
2. Avgerinos, K. I., Spyrou, N., Mantzoros, C. S. & Dalamaga, M. Obesity and cancer risk: Emerging biological mechanisms and perspectives. Metabolism 92, 121-135 (2019).
3. Calle, E. E., Rodriguez, C., Walker-Thurmond, K. & Thun, M. J. Overweight, Obesity, and Mortality from Cancer in a Prospectively Studied Cohort of U.S. Adults. N Engl J Med 348, 1625-1638(2003).
4. González-Muniesa, P. et al. Obesity. Nat Rev Dis Primers 3, 17034 (2017).
5. Castoldi, A., Naffah de Souza, C., Câmara, N. O. S. & Moraes-Vieira, P. M. The Macrophage Switch in Obesity Development. Front. Immunol. 6, (2016).
6. DeNardo, D. G. & Ruffell, B. Macrophages as regulators of tumour immunity and immunotherapy. Nat Rev Immunol 19, 369-382 (2019).
7. Ptacek, J. et al. Multiplexed ion beam imaging (MIBI) for characterization of the tumor microenvironment across tumor types. Lab Invest (2020) doi: 10.1038/s41374-020-0417-4.
8. Wisse, B. E., Kim, F. & Schwartz, M. W. PHYSIOLOGY: An Integrative View of Obesity. Science 318, 928-929 (2007).
9. Ishii, S., Kihara, Y. & Shimizu, T. Identification of T Cell Death-associated Gene 8(TDAG8) as a Novel Acid Sensing G-protein-coupled Receptor. Journal of Biological Chemistry 280, 9083-9087 (2005).
10. Wang, J.-Q. et al. TDAG8 Is a Proton-sensing and Psychosine-sensitive G-protein-coupled Receptor. J. Biol. Chem. 279, 45626-45633 (2004).
11. Radu, C. G. et al. Normal Immune Development and Glucocorticoid-Induced Thymocyte Apoptosis in Mice Deficient for the T-Cell Death-Associated Gene 8 Receptor. MCB 26, 668-677(2006).
12. Radu, C. G., Nijagal, A., Mclaughlin, J., Wang, L. & Witte, O. N. Differential proton sensitivity of related G protein-coupled receptors T cell death-associated gene 8 and G2A expressed in immune cells. Proceedings of the National Academy of Sciences 102, 1632-1637(2005).
13. Wirasinha, R. C. et al. GPR65 inhibits experimental autoimmune encephalomyelitis through CD4 +T cell independent mechanisms that include effects on INKT cells. Immunol Cell Biol 96, 128-136 (2018).
14. Onozawa, Y., Komai, T. & Oda, T. Activation of T cell death-associated gene 8 attenuates inflammation by negatively regulating the function of inflammatory cells. European Journal of Pharmacology 654, 315-319 (2011).
15. Sanderlin, E. J. et al. TDAG8 (GPR65) Inhibits Intestinal Inflammation in the DSS-Induced Experimental Colitis Mouse Model. http://biorxiv.org/lookup/doi/10.1101/496315 (2018) doi: 10.1101/496315.
16. Lassen, K. G. et al. Genetic Coding Variant in GPR65 Alters Lysosomal pH and Links Lysosomal Dysfunction with Colitis Risk. Immunity 44, 1392-1405 (2016).
17. Mogi, C. et al. Involvement of Proton-Sensing TDAG8 in Extracellular Acidification-Induced Inhibition of Proinflammatory Cytokine Production in Peritoneal Macrophages. J Immunol 182, 3243-3251 (2009).
18. Onozawa, Y. et al. Activation of T cell death-associated gene 8 regulates the cytokine production of T cells and macrophages in vitro. European Journal of Pharmacology 683, 325-331(2012).
19. Jin, Y. et al. Inhibition of interleukin-1B production by extracellular acidification through the TDAG8/cAMP pathway in mouse microglia. J. Neurochem. 129, 683-695 (2014).
20. Sin, W. C. et al. G protein-coupled receptors GPR4 and TDAG8 are oncogenic and overexpressed in human cancers. Oncogene 23, 6299-6303 (2004).
21. Li, S., Huang, S. & Peng, S.-B. Overexpression of G protein-coupled receptors in cancer cells: involvement in tumor progression. Int. J. Oncol. 27, 1329-1339 (2005).
22. Nassios, A. et al. Expression of proton-sensing G-protein-coupled receptors in selected skin tumors. Exp Dermatol exd.13809 (2018) doi: 10.1111/exd.13809.
23. Ihara, Y. et al. The G protein-coupled receptor T-cell death-associated gene 8 (TDAG8) facilitates tumor development by serving as an extracellular pH sensor. Proceedings of the National Academy of Sciences 107, 17309-17314 (2010).
24. Bohn, T. et al. Tumor immunoevasion via acidosis-dependent induction of regulatory tumor-associated macrophages. Nat. Immunol. 19, 1319-1329 (2018).

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method for the treatment of cancer in an overweight or obese individual, the method comprising:
administering an effective dose of an agent that blocks or inhibits GPR65 activation or decreases GPR65 expression in tumor associated macrophages, wherein tumor associated macrophage in the individual have increased anti-tumor activity after administration of the agent.

2. The method of claim 1, wherein the agent is an antibody that selectively binds to and inhibits GPR65 activation.

3. The method of claim 2, wherein the antibody selectively binds to an epitope in the first extracellular domain of GPR65.

4. The method of claim 1, wherein the agent is a small molecule drug that selectively binds to to and inhibits GPR65.

5. The method of claim 1, wherein the agent is an RNAi specific for GPR65.

6. The method of claim 4, wherein the RNAi targets a sequence of SEQ ID NO:1-16.

7. The method of claim 1, wherein the agent is an anti-sense RNA specific for GPR65.

8. The method of claim 1, comprising administering an effective dose of macrophages engineered to be deficient in GPR65 expression when tumor-associated.

9. The method claim 1, wherein administration is systemic.

10. The method of claim 1, wherein administration is localized to the tumor environment.

11. The method of claim 1, wherein the individual has a body mass index (BMI) of ≥25 kg/m².

12. The method of claim 1, wherein the individual has a body mass index (BMI) of ≥25 kg/m².

13. The method of any of claim 1, wherein the cancer is a solid tumor.

14. The method of claim 13, wherein the cancer is a carcinoma.

15. The method of claim 14, wherein the cancer is a colorectal carcinoma.

16. A method for the treatment of cancer in an individual, the method comprising:
administering an effective dose of an antibody that selectively binds to an epitope in the first extracellular domain of GPR65 and inhibits GPR65 activation.

17. The method of claim 16, wherein the individual has a body mass index (BMI) of <25 kg/m².

18. A composition for use in the methods of any of claim 1.


SEQUENCE LISTING (16 sequences)

SEQ ID NO: 1 - RNA, 25 nt, Homo sapiens
  source 1..25: mol_type = mRNA
tgaagaacagcatgacctggatcac

SEQ ID NO: 2 - RNA, 25 nt, Homo sapiens
  source 1..25: mol_type = mRNA
acagcatgacctggatcactatttg

SEQ ID NO: 3 - RNA, 25 nt, Homo sapiens
  source 1..25: mol_type = mRNA
atctttgtgattatagtcagcattc

SEQ ID NO: 4 - RNA, 25 nt, Homo sapiens
  source 1..25: mol_type = mRNA
caatattggatctctgtgtgtgtct

SEQ ID NO: 5 - RNA, 25 nt, Homo sapiens
  source 1..25: mol_type = mRNA
cttcagtttgtcactatcagattta

SEQ ID NO: 6 - RNA, 25 nt, Homo sapiens
  source 1..25: mol_type = mRNA
gttgatcggtatttggctgttgtct

SEQ ID NO: 7 - RNA, 25 nt, Homo sapiens
  source 1..25: mol_type = mRNA
cgatgccgaaaagtctaattttact

SEQ ID NO: 8 - RNA, 25 nt, Homo sapiens
  source 1..25: mol_type = mRNA
gaagagaatcataaaactacttgtc

SEQ ID NO: 9 - RNA, 25 nt, Homo sapiens
  source 1..25: mol_type = mRNA
cacagttacttttgtcttatgcttt

SEQ ID NO: 10 - RNA, 25 nt, Homo sapiens
  source 1..25: mol_type = mRNA
ttgtcttatgctttactccctttca

SEQ ID NO: 11 - RNA, 25 nt, Homo sapiens
  source 1..25: mol_type = mRNA
gagcatgctgtgaacttcgaagacc

SEQ ID NO: 12 - RNA, 25 nt, Homo sapiens
  source 1..25: mol_type = mRNA
gcaattctgggaagcgaacttacac

SEQ ID NO: 13 - RNA, 25 nt, Homo sapiens
  source 1..25: mol_type = mRNA
ctgggaagcgaacttacacaatgta

SEQ ID NO: 14 - RNA, 25 nt, Homo sapiens
  source 1..25: mol_type = mRNA
gcattaacaagtttaaattgtgttg

SEQ ID NO: 15 - RNA, 25 nt, Homo sapiens
  source 1..25: mol_type = mRNA
atatgatatgtggaatatattaaaa

SEQ ID NO: 16 - RNA, 25 nt, Homo sapiens
  source 1..25: mol_type = mRNA
gtcaccgagcttgaaagatttctat